(19) United States
(12) Patent Application Publication
(10) Pub. No.: US 2007/0049554 A1
(43) Pub. Date: Mar. 1, 2007

(54) Method for treatment or prevention of conditions caused by gram-positive bacteria

(76) Inventors: Daniel Levine, Briarcliff Manor, NY (US); Thomas S. Parker, Brooklyn, NY (US)

Correspondence Address: FULBRIGHT & JAWORSKI, LLP, 666 FIFTH AVE, NEW YORK, NY, 10103-3198, US

(21) Appl. No.: 11/510,094

(22) Filed: Aug. 25, 2006

Related U.S. Application Data
(60) Provisional application No. 60/712,075, filed on Aug. 29, 2005.

(51) Int. Cl. A61K 31/685; A61K 31/56

(52) U.S. Cl. 514/78; 514/169

(57) ABSTRACT

The treatment, prevention and prophylaxis of conditions such as sepsis, septic shock, systemic inflammatory response syndrome or “SIRS”, SIRS with organ dysfunction or failure, organ failure and organ dysfunction are described. These conditions are associated with infection by Gram-positive bacteria. The treatment involves the administration of compositions containing a phospholipid, a neutral lipid, and a bile acid or a bile acid salt.

### RELATED APPLICATION

This application claims priority of U.S. provisional application Ser. No. 60/712,075, filed Aug. 29, 2005 incorporated by reference in its entirety.

### FIELD OF THE INVENTION

This invention relates to the treatment of Gram-positive infections. More particularly, it relates to the treatment of such via administration of various compositions which act to neutralize and/or remove Gram-positive toxins from the organism, as well as prophylaxis utilizing these compositions. Most preferably, these compositions contain a bile acid or bile acid salt, such as a cholanoic acid or cholanoic acid salt, a neutral lipid, such as a triglyceride, and a phospholipid, such as phosphatidylcholine, and are used for treatment or prophylaxis of conditions caused by Gram-positive bacteria, such as, but not being limited to, sepsis, septic shock, systemic inflammatory response syndrome (SIRS), SIRS with organ dysfunction and/or failure, organ failure, and organ dysfunction.

### BACKGROUND OF THE INVENTION

Normal serum contains a number of lipoprotein particles which are characterized according to their density, namely, chylomicrons, very low density lipoprotein (VLDL), low density lipoprotein (LDL) and high density lipoprotein (HDL). They are composed of free and esterified cholesterol, triglycerides, phospholipids, several other minor lipid components, and protein. VLDL transports energy, in the form of triglycerides, to the cells of the body for storage and use. As triglycerides are delivered, VLDL is converted to LDL. LDL transports cholesterol and other lipid soluble materials to the cells in the body, while HDL transports excess or unusable lipids to the liver for elimination. Normally, these lipoproteins are in balance, ensuring proper delivery and removal of lipid soluble materials. Abnormally low HDL can cause a number of diseased states as well as constitute a secondary complication in others.

Under normal conditions, a natural HDL is a solid particle with its surface covered by a phospholipid monolayer that encloses a hydrophobic core. Apolipoprotein A-I and A-II attach to the surface by interaction of the hydrophobic face of their alpha helical domains. In its nascent or newly secreted form, the particle is disk-shaped and accepts free cholesterol into its bilayer. Cholesterol is esterified by the action of lecithin:cholesterol acyltransferase (LCAT) and is moved into the center of the disk. The movement of cholesterol ester to the center is the result of space and solubility limitations within the bilayer. The HDL particle “inflates” to a spheroidal particle as more and more cholesterol is esterified and moved to the center. Cholesterol ester and other water insoluble lipids which collect in the “inflated core” of the HDL are then cleared by the liver.

Anantharamaiah, in Segrest et al., Meth. Enzymol. 128:627-647 (1986) describes a series of peptides which form “helical wheels”, as a result of the interaction of the amino acids in the peptide with each other. Such helical wheels present a nonpolar face, and a polar face in their configuration. The reference shows, generally, that peptides can replace apoproteins in these particles.

Jonas et al., Meth. Enzym. 128A: 553-582 (1986) have produced a wide variety of reconstituted particles resembling HDL. The technique involves the isolation and delipidation of HDL by standard methods (Hatch et al., Adv. Lip. Res. 6: 1-68 (1968); Scanu et al., Anal. Biochem. 44:576-588 (1971)) to obtain apo-HDL proteins. The apoproteins are fractionated and reconstituted with phospholipid and with or without cholesterol using detergent dialysis.

Matz et al., J. Biol. Chem. 257(8): 4535-4540 (1982) describe a micelle of phosphatidylcholine, with apolipoprotein A1. Various ratios of the two components are described, and it is suggested that the described method can be used to make other micelles. It is suggested as well to use the micelles as an enzyme substrate, or as a model for the HDL molecule. This paper does not, however, discuss application of the micelles to cholesterol removal, nor does it give any suggestions as to diagnostic or therapeutic use.

Williams et al., Biochem. & Biophys. Acta 875:183-194 (1986) teach phospholipid liposomes introduced to plasma which pick up apoproteins and cholesterol. Liposomes are disclosed, which pick up apoprotein in vivo, as well as cholesterol, and it is suggested that the uptake of cholesterol is enhanced in phospholipid liposomes which have interacted with, and picked up apoproteins.

Williams et al., Persp. Biol. & Med. 27(3): 417-431 (1984) discuss lecithin liposomes as removing cholesterol. The paper summarizes earlier work showing that liposomes which contain apoproteins remove cholesterol from cells in vitro more effectively than liposomes which do not contain it. They do not discuss in vivo use of apoprotein containing liposomes or micelles, and counsel caution in any in vivo work with liposomes.

U.S. Pat. Nos. 5,506,218; 5,344,822; 5,614,507; 5,587,366; 5,674,855 and all of which are incorporated by reference, describe how formulations containing a phospholipid, such as phosphatidylcholine, a natural lipid, such as a triglyceride, and a bile acid or a bile acid salt, such as a cholanoic acid or cholanoic acid salt, such as sodium cholate, function to prevent or alleviate Gram-negative bacterial infections, such as infection via S. typhimurium, via inactivation of the lipid A anchored molecule “LPS”.

In U.S. Pat. No. 5,128,318 the disclosure of which is incorporated by reference, it was taught that reconstituted particles containing both an HDL associated apolipoprotein and a lipid capable of binding an endotoxin to inactivate it could be used as effective materials for alleviating endotoxin caused toxicity.

It has now been found, quite surprisingly, that phospholipids may be used alone, or in combination with additional materials, such as neutral lipids, bile acid salts, etc., as effective agents to alleviate and/or prevent Gram-positive infections. It is especially preferred to use phosphatidylcholines (“PC” hereafter), either alone, or in combination with other phospholipids, such as sphingolipids, in compositions which are essentially free of peptides and proteins, such as apolipoproteins or peptides derived therefrom. Neutral lipids such as mono-, di-, and triglycerides may be combined with the phospholipids, as long as the total amount of neutral lipids is below certain weight percents when the compositions are used in the form of an intravenous bolus. When used in other forms of administration, such as intravenously for example, by continuous infusion, the weight percents are not so critical, but are desirable. Bile acids or bile acid salts, such as cholates, e.g., sodium cholate, may be combined with these other two components to produce particularly efficacious formulations.

Particularly preferred embodiments of the invention are those compositions where the neutral lipid is a triglyceride, a cholesterol ester, or a mixture of cholesterol ester and triglycerides.

The efficacy of bile acids and bile acid salts, such as cholates, in the treatment, prophylaxis, and/or prevention, of Gram-positive infections and/or neutral lipids, such as a phosphatidylcholine, and/or a triglyceride is shown herein. These bile acids may be used alone, or in combination with one or more phospholipids, and/or neutral lipids, such as a phosphatidylcholine, and/or a triglyceride. These compositions can be used in treatment or prophylaxis of conditions, including but not being limited to, those set forth supra, preferably using compositions such as those set forth supra, even more preferably, in the form of an emulsion.

The invention is described in greater detail, as follows:

### DETAILED DESCRIPTION OF PREFERRED EMBODIMENTS

### Example

In the experiments which follow, it was determined that an emulsion containing phosphatidylcholine, triglyceride, and sodium cholate, was useful for clearing Gram-positive toxins from blood.

An emulsion was prepared, containing a solution of phosphatidylcholine (“PC”) hereafter, at 99.7 mg/ml, 18 mM sodium cholate and triglycerides (TG) which amounted to 7.5% of the weight of the total lipid weight in the emulsion in a aqueous solution of glycerol (2.6%).

As a control, a 2.6% solution of glycerol was used.

The emulsion and the control were both diluted, 1:10, and were added to test solutions of EDTA treated whole blood, at 50% dilution, to which varying dilutions of a 5 mg/ml solution of lipoteicholic acid (“LTA”) obtained from B. subtilis were added.

The samples were mixed and incubated for 4 hours at 37° C., after which they were quenched on ice. Samples were centrifuged (1000 RPMs, 2000×g), and plasma tumor necrosis factor (“TNF”) was measured, using a standard, commercially available ELISA.

The results, which are presented in Table 1 (emulsion), and Table 2 (control), show the efficacy of the emulsions in removing the toxin from blood.

TABLE 1
EMULSION

| LTA | TNF |
|---|---|
| ng/ml | pg/ml |
| 0.001 | 0 |
| 0.010 | 0 |
| 0.100 | 0 |
| 0.300 | 0 |
| 1.000 | 4.531 |
| 3.000 | 0 |
| 10.000 | 0 |
| 100.000 | 15.15 |

TABLE 2
GLYCEROL CONTROL

| LTA | TNF |
|---|---|
| ng/ml | pg/ml |
| 0.001 | 0 |
| 0.010 | 0 |
| 0.100 | 44.19 |
| 0.300 | 8.081 |
| 1.000 | 66.41 |
| 3.000 | 58.07 |
| 10.000 | 39.4 |
| 100.000 | 290.7 |

The foregoing examples detail the invention which involves, in one aspect, a method of treatment or prevention of sepsis, septic shock, systemic inflammatory response syndrome (SIRS), SIRS with organ dysfunction/failure, organ failures and organ dysfunction caused by Gram-positive bacteria.

The examples also show that administration of a member of the family of bile acids or bile acid salts, such as a cholanoic acid or a cholanoic acid salt can also be used in combination with the phospholipid and neutral lipid, or with the phospholipid alone for, e.g., the prophylaxis, alleviation, prevention or treatment of Gram-positive bacterial infections. Thus, peptide and protein free compositions containing one, or both, of a bile acid/bile acid salt and a phospholipid may be used to treat such infections. Cholanoic acids are described by, e.g., Hofmann, Hepatology 4(5): 4S-14S (1984), incorporated by reference. Attention is drawn in particular to page 5S, FIGS. 1 and 2, incorporated by reference, showing the structures characteristic of the cholanoic acids.

The subject being treated is preferably a human, but the practice of the invention is equally applicable in a veterinary context as well.

“Alleviation” as used herein refers to treatment to ease the burden of the infection caused by any of the various toxins produced by Gram-positive bacteria (e.g., B. subtilis). Prophylaxis may be accomplished by administering the agent at a point where the subject is in or about to be in, a situation where exposure to Gram-positive bacteria may result. Classically, this occurs during surgery. Thus, a subject who is about to experience a surgical procedure may have the active ingredient administered preparatory to the procedure.

The effective amount of phospholipid and bile acid combination necessary for treatment of the subject can vary. In general, a total dose up to from about 200 mg to about 800 mg of phospholipid per kilogram of body weight of the subject is preferred, although the amount may drop, or increase, depending upon the severity of the infection or the degree of risk in the context of the prophylaxis. For bile acids and salts, such as the cholanoic acids and their salts, a dose of from about 10 mg to about 300 mg/kg of body weight, more preferably 15 mg to about 275 mg per kg of body weight is used.

It is desirable to administer the bile acid/bile acid salt and phospholipids in compositions which also contain neutral lipids, but this is not necessary, as neutral lipid free emulsions of phospholipids are also envisioned. The desirability of combined administration of the phospholipids with neutral lipids results from the fact that the neutral lipids and phospholipids associate into particles which resemble the lipoproteins, but differ therefrom in that they contain no protein or peptide components, which are of course, always present in the lipoproteins.

Especially desirable forms of treatment are those where the phospholipid is a phosphatidylcholine, such as egg yolk phosphatidylcholine, soy based phosphatidylcholine or a sphingolipid. For the bile acid/bile acid salt, preferred are cholanoic acid and/or its salts, such as sodium cholate, sodium deoxycliolate, and sodium chenodeoxycholate. With respect to the neutral lipids, it is preferred to use a cholesterol ester or a triglyceride, but other neutral lipids, such as squalene or other hydrocarbon oils, di- and mono-glycerides and antioxidants such as vitamin E may also be used.

The form in which the compositions may be administered can vary, with a bolus or other intravenous forms being especially preferred. When a bolus form is used, and the composition contains triglyceride, e.g., some care must be given in dosing. It is fairly well known that triglycerides are toxic if administered in too large an amount. The artisan of ordinary skill, however, can easily formulate the compositions so that the risk of triglyceride poisoning is reduced, or eliminated. In general, when a bolus form is used, the compositions should contain no more than about 80 percent by weight of triglyceride or other neutral lipid, preferably no more than 70 percent by weight. Most preferably, the compositions should contain no more than about 50 percent by weight, of neutral lipid, when a bolus is administered.

When non-bolus forms are employed, however, such as other intravenous forms, the risk of poisoning is decreased. Nonetheless, the ranges delineated supra are preferred for intravenous, or other forms of administration, although it must be understood that they are not required. Preferably, a dose of up to about 200 mg per kg of body weight of bile acid/bile acid salt or phospholipid is administered. Administration of up to about 800 mg/kg is also feasible. Doses are general, however, and will vary depending upon the subject and the form of administration.

As indicated, supra, the protein and peptide free formulations require that at least one phospholipid or bile acid/bile acid salt be present. For phospholipids, it is preferred that at least one neutral lipid, such as a triglyceride, diglyceride, or monoglyceride be present. The compositions may include additional materials such as sterols (e.g., cholesterol, .beta.-sitosterol), esterified or unesterified lipids (e.g., cholesterol ester or unesterified cholesterol), hydrocarbon oils such as squalene, antioxidants such as vitamin E, but these are not required. Of course, more than one phospholipid, and/or more than one neutral lipid may be used in any such formulation. When combinations of neutral lipid and phospholipid are used, the neutral lipid should be present at from about 3% up to about 50% by weight relative to the total amount of lipid in the composition.

In the case of the bile acid/bile acid salts, these may be used separately, or in combination with a phospholipid, a neutral lipid, or both. With respect to these additional materials (e.g., phospholipids and neutral lipids), preferred species are those discussed and mentioned supra. Optional additional ingredients include those listed supra.

Also a part of the invention is the use of compositions in treating specific conditions associated with infection by bacteria, such as those conditions described supra. These compositions preferably contain, by weight percent, from about 5% to about 30% by weight bile acid/bile acid salt, from about 3% to about 50% by weight neutral lipid, and from about 10% to about 95% by weight of phospholipid, and are protein and peptide free. Preferably, these compositions are in the form of an emulsion. Especially preferred are compositions containing from about 10-15% by weight of bile acid/bile acid salt, from about 5% to about 10% by weight of neutral lipid, and the balance of the composition being phospholipid. “Protein and peptide free,” as this phrase is used herein, refers to compositions which do not contain sufficient protein or peptide, or both, to treat or prevent conditions such as those set forth herein, although residual, insufficient amounts of protein or peptide may be present.

It should be noted that these weight percentages are relative to compositions consisting of three components. When the three-component system is combined with, e.g., a carrier, adjuvant, or optional ingredients such as those discussed supra, the percentage by weight relative to the entire composition will drop; however, the ratios of each component, relative to each other, will remain the same. It is to be borne in mind that such therapeutic compositions are always substantially protein free and peptide free.

In the case of compositions which do not contain a bile acid or a bile acid salt, such protein free, peptide free compositions contain, preferably, at least about 3% by weight of a neutral lipid, up to about 50% by weight neutral lipid, the balance being at least one phospholipid. Preferably, the neutral lipid is a triglyceride, but may be any of the additional neutral lipids discussed supra. Also, the phospholipid is preferably a phosphatidylcholine.

The compositions of the invention are efficacious in the treatment or prevention of conditions caused by Gram-positive bacteria, including, but not being limited to conditions such as, but not being limited to, sepsis, septic shock syndrome, systemic inflammatory response syndrome or “SIRS”, SIRS with organ dysfunction or failure, organ failure, and/or organ dysfunction caused by Gram-positive bacteria.

Other aspects of the invention will be clear to the skilled artisan and need not be reiterated here.

It will be understood that the specification and examples are illustrative, but not limitative, of the present invention, and that other embodiments within the spirit and scope of the invention will suggest themselves to those skilled in the art.

What is claimed is:

1. A method for treating or preventing a condition caused by Gram-positive bacteria, selected from the group consisting of: sepsis, septic shock, systemic inflammatory response syndrome (SIRS), SIRS with organ dysfunction or failure, organ failure, and organ dysfunction, comprising administering, intravenously to a subject in need thereof an effective amount of a protein free, peptide free, composition containing a sufficient amount of a bile acid or bile acid salt and a phospholipid to treat or to prevent the condition in said subject.

2. The method of claim 1, wherein said bile acid or bile acid salt is a cholanoic acid or cholanoic acid salt.

3. The method of claim 1, wherein said phospholipid is a phosphatidylcholine.

4. The method of claim 1, wherein said composition further comprises a neutral lipid.

5. The method of claim 4, wherein said neutral lipid is a triglyceride.

6. The method of claim 2, wherein said bile acid is a cholanoic acid.

7. The method of claim 1, wherein said bile acid salt is a cholate salt.

8. The method of claim 7, wherein said cholate salt is a sodium cholate.

9. The method of claim 2, wherein said composition further comprises a neutral lipid.

10. The method of claim 9, wherein said phospholipid is a phosphatidylcholine and said neutral lipid is a triglyceride.

11. The method of claim 1, comprising administering said composition intravenously.

12. The method of claim 1, wherein said Gram-positive bacteria comprise Bacillus subtilis.

13. The method of claim 1, wherein said Gram-positive bacteria produce lipoteicholic acid (LTA).

14. The method of claim 1, wherein said composition is in the form of an emulsion.

15. The method of claim 4, wherein said composition comprises from about 5% to about 30% by weight of bile acid or bile acid salt, from about 3% to about 50% by weight of neutral lipid, and from about 10% to about 95% by weight phospholipid.

16. A method for treating or preventing an infection by Gram-positive bacteria comprising administering, intravenously to a subject in need thereof an effective amount of a protein free, peptide free, composition containing a sufficient amount of a bile acid or bile acid salt and a phospholipid to treat or to prevent the infection in said subject.
